(19) United States
(12) Patent Application Publication
(10) Pub. No.: US 2003/0055404 A1
(43) Pub. Date: Mar. 20, 2003

(54) Endoscopic rotary abraders

(76) Inventor: Timothy E. Moutafis, Gloucester, MA (US)

Correspondence Address: HydroCision, Inc., Att'n. Francis Kirkpatrick, 100 Burtt Rd, Andover, MA, 01810, US

(21) Appl. No.: 10/245,287

(22) Filed: Sep. 17, 2002

Related U.S. Application Data
(60) Provisional application No. 60/322,815, filed on Sep. 17, 2001.
(60) Provisional application No. 60/322,855, filed on Sep. 17, 2001.
(60) Provisional application No. 60/322,856, filed on Sep. 17, 2001.
(60) Provisional application No. 60/322,857, filed on Sep. 17, 2001.

(51) Int. Cl. A61B 1/00; A61B 17/14; A61B 17/32; A61M 1/00

(52) U.S. Cl. 604/540; 606/180; 600/104

(57) ABSTRACT

An improved design for a powered abrading or cutting instrument for surgery is described. A basic instrument comprising a burr, a driveshaft, a support tube or shaft for the driveshaft, and a bearing between the driveshaft and the support is further provided with an external tube. The external tube provides one or more of drainage, irrigation, and provision of a sheath function. Some embodiments of the improved design can remove debris without requiring suction or other mechanical assistance, and have improved resistance to clogging.

### RELATED APPLICATIONS

This non-provisional application claims the benefit under Title 35, U.S.C. § 119(e) of co-pending U.S. provisional application serial Nos. 60/322,815, 60/322,855, 60/322,856, and 60/322,857, filed Sep. 17, 2001. U.S. provisional application Nos. 60/322,815, 60/322,855, 60/322,856, and 60/322,857 are each incorporated herein by reference.

### FIELD OF THE INVENTION

This invention relates to improved designs for rotary abraders for use in surgery.

### BACKGROUND

The use of powered rotary cutters and abraders is useful in surgery and other medical procedures, particularly when treatment of hard tissues, such as bone, is conducted. Rotary drills and polishers are familiar in dentistry, for example. A current challenge is to provide cutting instruments that can be used in minimally invasive surgery, such as arthroscopy, laparoscopy, and other endoscopic types of procedures. These are collectively called “endoscopy” or “endoscopic” herein, including any minimally invasive procedure conducted through a small puncture, or a narrow natural opening. Typically, a puncture made for insertion of endoscopic instruments is made with an obdurator, a trocar or a cannula. Generally, rotary abraders in current endoscopic use have a diameter in the range of about an inch, or less. Such instruments can also be used in open surgery if desired.

The key problems in endoscopy are providing instruments that are small enough to pass through a trocar; to provide for debris removal from the site; and to provide good tactile handling properties, to facilitate accurate tissue removal by the surgeon. Some of these problems have been addressed by existing instruments. The design of U.S. Pat. No. 4,842,578, for instance, provides an instrument that can be passed through a trocar, and the instrument provides for debris removal by vacuum assistance, via the interior of its drive shaft. Suction is typically required in this design because the relatively low-speed cutter provided in the prior art produces large particles that can clog a debris-removing means. Other designs include U.S. Pat. Nos. 3,937,222, 3,384,085, and 3,990,453. Some of these older designs do not provide for debris removal systems.

In our co-pending application U.S. Ser. No. 09/480,500, hereby incorporated by reference, we disclose a rotary abrader/cutter (hereafter, collectively referred to by the term “burr”, used for “abrasion”, which also includes cutting, grinding, shaving, polishing, coring, and similar surgical maneuvers) that is, in preferred embodiments, driven by a liquid jet powered rotor. The burr tends to run at high speed in these instruments, providing rapid tissue removal combined with much smaller debris fragments compared to other endoscopic instruments. While some known designs for supporting the abrader or burr can potentially be used in combination with the liquid jet powered rotor driven instruments, there remains a need for improved designs to more fully take advantage of the improved properties provided by the liquid jet powered rotor drive. An improved design in several embodiments is described in this application.

### SUMMARY OF THE INVENTION

Rotary abrading and cutting devices (“devices”, herein) are provided with alternative methods for management of fluids and debris, and for providing lateral support to the rotating shaft and burr. Preferred devices have in common features that provide at least one method of removal of debris that is less likely to plug than most prior art devices. In particularly preferred embodiments, debris can be removed without requiring an external vacuum. Preferred devices provided according to the invention incorporate at least a cutting, polishing or abrading head (a “burr”); a shaft carrying the burr, which shaft may be hollow or solid or a combination; an element, most commonly tubular but in some embodiments optionally solid, supporting the shaft via a distal bearing; means for sheathing the burr so that it does not abrade tissue that is adjacent to the tissue to be abraded; and means for removal of fluid and debris from the site (“evacuation”, but not necessarily requiring a vacuum to be operative).

In one embodiment, the driveshaft (“shaft”) of a cutting or abrading head is supported by a distal bearing and a tube. A “distal” bearing herein is a bearing supporting the shaft at a location on the shaft that is proximal to the burr and nearer to the burr than to the proximal end of the shaft. In preferred embodiments, the distal bearing(s) is both near the burr and proximal to it. An evacuation pathway and a sheath function are supplied, preferably, by a separate tube external to the support tube (which separate tube external to the support tube is hereinafter referred to as an “external tube”), providing a large space to use for evacuation. The external tube is typically maintained in concentricity to the support tube by standoffs or similar features.

In a second embodiment, the support tube is flared in the vicinity of the burr, distal of the support bearing, to provide a sheath function. An external tube, similar to that of the first embodiment, is, preferably, provided for evacuation. Optionally, holes are provided in the proximal region of the sheath to efficiently convey debris to the external tube.

In a third embodiment, the support tube is replaced by a support shaft internal of the shaft carrying the abrading head. A distal bearing is provided on the distal portion of the support shaft between the support shaft and the drive shaft. An external tube, in preferred arrangements, provides a sheath function, and optionally a pathway for debris removal. Alternatively, a pathway can be provided internally of the support shaft for debris removal.

In each of these embodiments, the outer tube can be provided with ribs or “feet” to provide spacing from the next inward element, which may be the support tube or the shaft carrying the burr.

Other advantages, novel features, and uses of the invention will become more apparent from the following detailed description of non-limiting embodiments of the invention when considered in conjunction with the accompanying drawings, which are schematic and which are not intended to be drawn to scale. In the figures, each identical, or substantially similar component that is illustrated in various figures is typically represented by a single numeral or notation. For purposes of clarity, not every component is labeled in every figure, nor is every component of each embodiment of the invention shown where illustration is not necessary to allow those of ordinary skill in the art to understand the invention. In cases where the present specification and a document incorporated by reference include conflicting disclosure, the present specification shall control.

### BRIEF DESCRIPTION OF THE FIGURES

FIG. 1 is a cross-sectional illustration showing a distal portion of a surgical instrument with a prior art shaft support design;

FIG. 2 is a cross-sectional illustration showing a distal portion of a surgical instrument with another prior art shaft support design;

FIG. 3 is a cross-sectional illustration showing a first embodiment of the distal portion of a surgical instrument according to the invention;

FIG. 4 is a cross-sectional illustration showing a second embodiment of the distal portion of a surgical instrument according to the invention;

FIG. 4a is a cross-sectional illustration of the distal portion of the outermost external tube of FIG. 4, taken along lines a-a;

FIG. 5 is a cross-sectional illustration showing an embodiment of the distal portion of a surgical instrument according to the invention similar to that of FIG. 4, except including evacuation openings in the sheath region of the support tube; and

FIG. 6 is a cross-sectional illustration showing a third embodiment of the distal portion of a surgical instrument according to the invention;

FIG. 7 is a cross-sectional illustration showing an alternative embodiment of the distal portion of a surgical instrument according to the invention similar to that of FIG. 6.

### DETAILED DESCRIPTION OF THE INVENTION

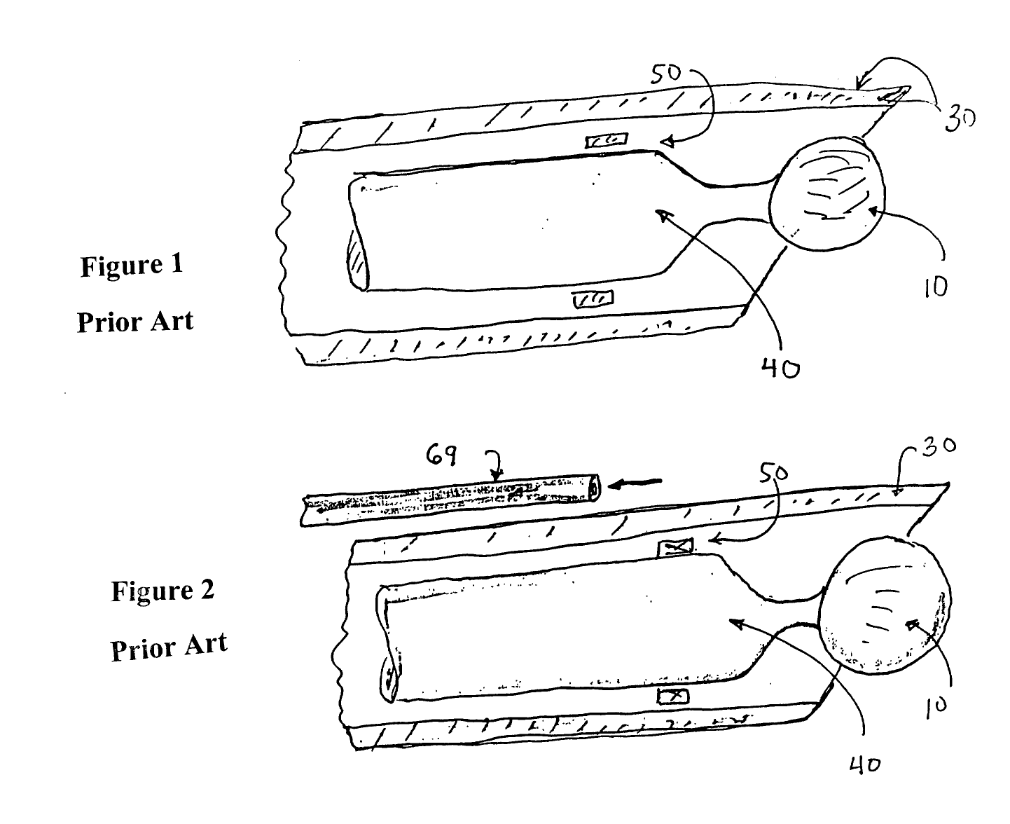

A known abrader, for example that of Hall (U.S. Pat. No. 3,384,085), is illustrated schematically in FIG. 1. The figure illustrates the distal (operative) portion of an abrading instrument for use with a drive. The abrading head or “burr” 10, which may have any of a large variety of shapes and profiles known in the art or similar thereto, is mounted on a shaft 40, which may be solid, hollow, or a combination, that is in turn driven by a source of rotary motion, for example a liquid jet powered rotor, a turbine, or an electric motor, at the proximal end of the instrument (not illustrated; see the cited references for views of an entire apparatus.)

The shaft 40 is enclosed by a tubular support 30, which supports the shaft against lateral deflection by at least one bearing 50 in the distal portion of the device. Simple journal-type bearings made of low-friction materials are generally adequate; other bearing types are useable, including roller bearings and the like, as would be apparent to those skilled in the art. It is preferable to provide sufficient sealing to prevent debris-containing fluid from being drawn from the region of the abrader 10 into the bearing 50, to minimize friction.

In the illustrated embodiment, the tubular support 30 also acts as a sheath. The sheath region extends beyond the bearing 50 and the abrading head 10, and shields the abrader 10 from tissue contact except where the sheath portion of the support tube 30 is cut away to provide a controlled zone of abrasion, cutting, etc.

Evacuation is not directly supplied in this simple design. In FIG. 2, a separate evacuation lumen 69 is illustrated. Such a drain could be provided in association with the device, or it could be separately inserted into the operating space.

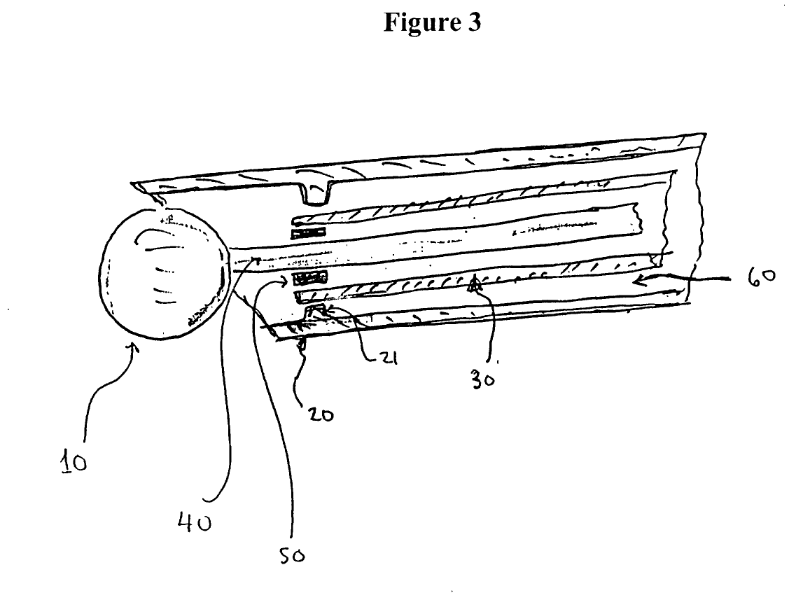

FIG. 3 shows a first embodiment of the invention. In this embodiment, the burr 10, shaft 40 and bearing 50 are similar in configuration to the instrument illustrated in FIG. 1. In the present embodiment, however, the support 30 is altered to terminate proximally of the burr 10. The tubular support 30 is surrounded by an external tube 20 that acts as a sheath. The external tube 20 extends beyond the outer support, and shields the burr 10 from tissue contact except where the external tube 20 is cut away to provide a controlled zone of abrasion, cutting, etc. The external tube 20 is maintained in a selected position relative to the burr 10 by stand-offs 21. These may be of any appropriate design, but preferably are longitudinal ribs, or are discrete “feet”, so that most of the annular area 60 bounded by the external tube 20 and the tubular support 30 is open. This allows the space between the sheath and the support to be used either as an outlet for debris, or as an inlet for lavage of the site of operation, or both. If multiple ribs are provided, both operations could be performed simultaneously in space 60. The feet, ribs or other standoff elements may be provided in any convenient way. For example, and without limitation, they may be molded into a tube, or pressed into or other wise formed in a preformed tube, or supplied by separately formed pieces inserted into a tube, and preferably held in place by adhesion, welding, press fitting, or other conventional methods for retaining a piece in place in a tube. However, the external tube 20 provides substantially no lateral support for the shaft or the burr. Support tube 30 and bearing 50 are the primary supports preventing lateral deflection of the burr 10 and shaft 40, and any support to support tube 30 provided by external tube 20 via standoffs 21 is incidental, i.e., the degree of deflection of the burr or shaft under side loading is not significantly affected by the presence or absence of external tube 20.

Because the debris is typically small in diameter relative to that in the prior art, (because of higher speeds available from the liquid jet powered rotor drive means of preferred instruments), and because the outlet space 60 is relatively large, suction is not typically required for debris removal with this design. In particular, a vacuum source or a suction or aspiration source is not typically needed. A slight positive pressure, for example provided by elevation of a bag of saline solution used for irrigating the site, can be sufficient to provide flow through the outlet space 60. Significant debris-removal impulse can also be provided by the particular design of the burr 10, as known to those skilled in the art, even in the absence of a hydrostatic head in the operation site.

An additional improvement provided by this design is the ability, in some embodiments, to vary the position of the external tube 20 with respect to the burr 10, by sliding or rotating the sheath with respect to the support tube. A simple bellows or similar means at the proximal end of the sheath (not illustrated) would supply the needed range of motion. Movement of the external tube 20 can be manual, as the proximal region of the sheath, near the driving device, is normally outside of the entry point into the patient; or controls operable from a handle of the device, or other location, can be provided.

An additional advantage of the design is that the external tube 20 can be made of plastic, allowing direct visual or fluoroscopic observation of the position of the abrader. The tip of the external tube 20 can be made to be radio-opaque or visible (e.g., by dye) if desired.

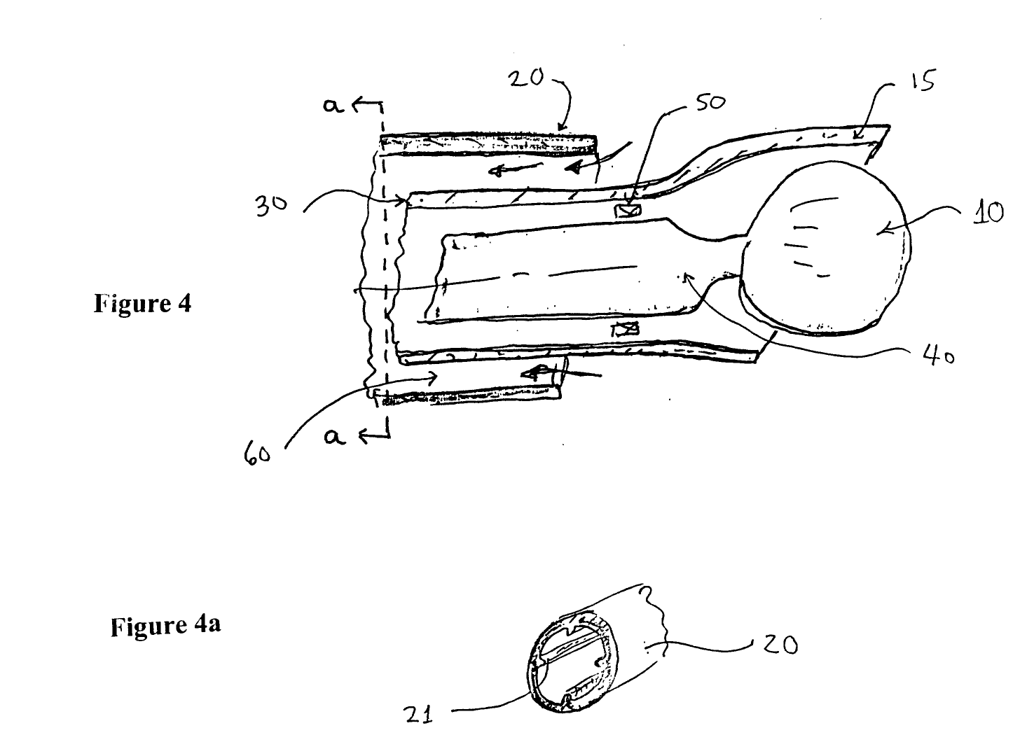

FIG. 4 shows a second embodiment of the invention, as an alternative version of the first embodiment. The burr 10, which may have any of a large variety of shapes and profiles, is mounted on a shaft 40, which may be solid, hollow, or a combination, that is in turn driven by a source of rotary motion, for example a turbine, a liquid jet powered rotor, or an electric motor, at the proximal end of the instrument (not illustrated).

As in FIG. 3, the shaft 40 is enclosed by a tubular support 30, which supports the shaft against lateral deflection via at least one bearing 50 in the distal portion of the device. It is preferable to provide sufficient sealing to prevent debris-containing fluid from being drawn from the region of the burr 10 into the bearing 50, to minimize friction.

The tubular support 30 is expanded at the distal end into a sheath region 15. The sheath extends beyond the tubular support 30 laterally and distally, and shields the burr 10 from tissue contact except where it is cut away to provide a controlled zone of abrasion, cutting, etc.

A debris removal channel is formed by an external tube 20. The external tube 20 is maintained in a selected position relative to the tubular support 30 by longitudinal fins or discrete “feet” 21, illustrated in FIG. 4a, which is a perspective view of a cross section of external tube 20. Returning to FIG. 4, the fins or feet 21 ensure that most of the annular area 60 bounded by the external tube 20 and the tubular support 30 is open. This allows the space between the external tube 20 and the support tube 30 to be used either as an outlet for debris, or an inlet for lavage of the site of operation, or both. If multiple ribs 21 are provided, both operations could be performed simultaneously. The external tube 20 may be moveable, as described in the first embodiment.

Again, because the debris is typically small in diameter, and the outlet space 60 is relatively large, suction is not typically required for debris removal with this design. In particular, a vacuum source or a suction or aspiration source is not typically needed. A slight positive pressure, for example provided by elevation of a bag of saline solution used for irrigating the site, can be sufficient to provide flow through the outlet space 60. As previously mentioned, some debris-removal impulse can also be provided by the design of the burr 10 even in the absence of a hydrostatic head in the operation site.

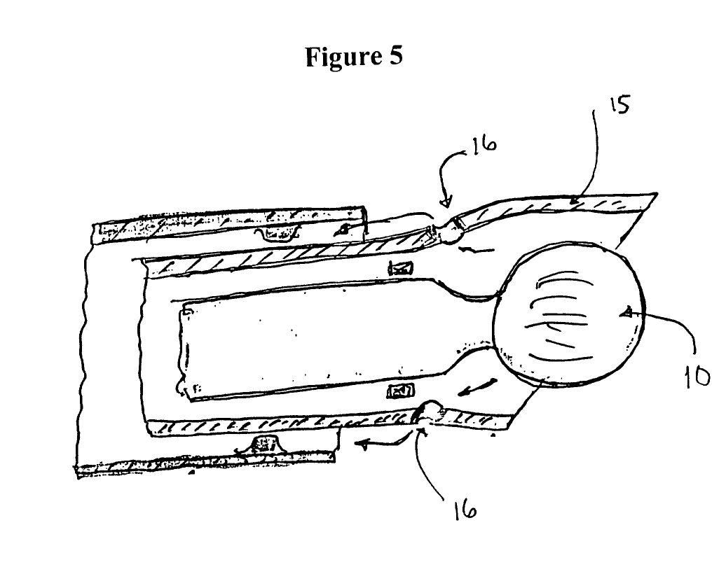

FIG. 5 shows a variant of the apparatus of FIG. 4 in which openings 16 are created in the sheath region 15 of the support tube 30 to provide more direct removal of the debris from the region around the burr 10 to the debris removal space 60. Debris may also pass outside the sheath, as in FIG. 4.

As in FIGS. 3 or 4, an additional improvement provided by this design is the ability, in some embodiments, to vary the position of the external tube 20 with respect to the burr 10 by sliding or rotating the outer tube with respect to the support tube. This variation can allow control of the location from which debris-containing fluid is removed, thereby helping to control the visual clarity of the operating field.

An additional advantage of the design is that the external tube 20 can be made of plastic, for example by extrusion, thereby allowing direct visual or fluoroscopic observation of the position of the abrader. The tip of the external tube 20 can be made to be radio-opaque or visible (e.g., by dye) if desired.

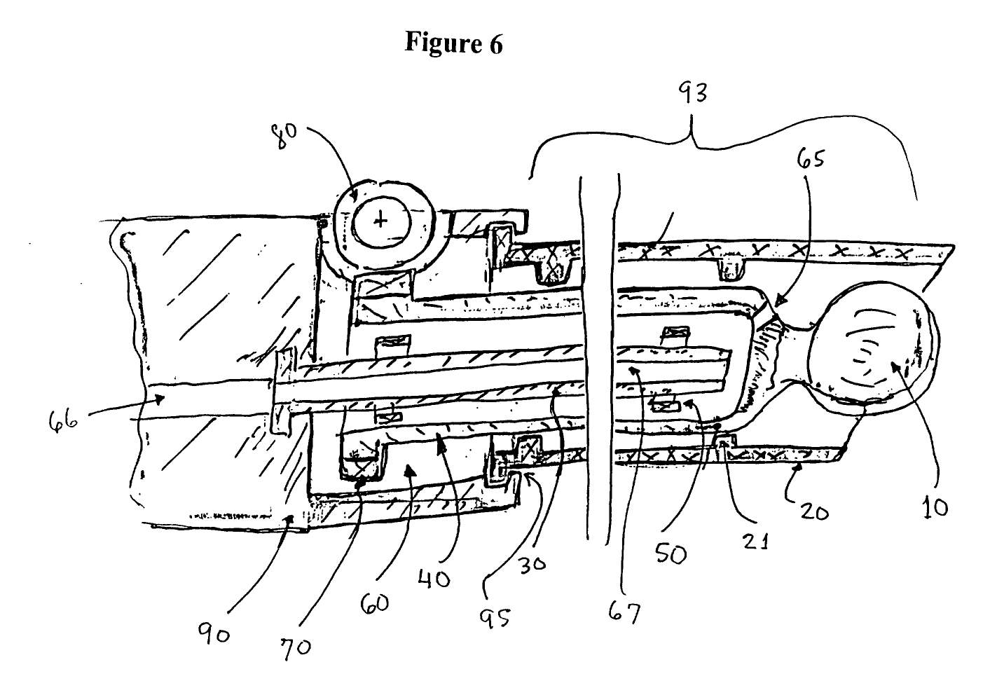

A third embodiment of the invention is illustrated schematically in FIG. 6. The figure illustrates the distal (operative) portion of an improved abrading instrument. The burr 10, which may have any of a large variety of shapes and profiles, is mounted on a shaft 40, which in this embodiment is hollow, that is in turn driven by a source of rotary motion, for example a turbine, liquid jet powered rotor, or an electric motor, (not shown) at the proximal end of the instrument (to the left of the portion of the instrument illustrated in the drawing). Here a portion of the source of rotary motion is shown, namely, a step-down worm gear 80, which is driven by a primary source (not illustrated), and which, in turn, drives a gear 70 attached to the shaft 40. As illustrated, the distal portion 93 of the device can be detached from a handpiece body 90 carrying the primary source of rotational energy by a latch or other connector 95, but the distal end 93, in other embodiments, could also be permanently affixed to the handpiece body. The exact method of connection of the abrading element and the drive and handpiece is not critical, and any of the many known methods illustrated in the art for connecting abrading devices to handpieces is potentially of use in the invention.

The shaft 40 is supported internally by a support 30, which can be hollow or solid. The support 30 is affixed to a handpiece body 90 or other supporting element, so that it provides support to the shaft 40 via bearings 50, typically at least one in the distal region of the support/shaft interface, or by other means of providing support while minimizing friction. Simple journal-type bearings made of low-friction materials are generally adequate; other bearing types are useable, including roller bearings and the like, as would be apparent to those skilled in the art. It is preferable to provide sufficient sealing to prevent debris-containing fluid from being drawn from the region of the burr 10 into the bearing 50, to minimize friction.

The shaft 40 is surrounded by an external tube 20. As in previous embodiments, the external tube 20 is not a support to prevent deflection of burr 10 or shaft 40; that function is provided by support tube 30 and bearing 50. The external tube 20 extends beyond the support 30 and the shaft 40 to provide a sheath, and shields the burr 10 from tissue contact except where the external tube 20 is cut away to provide a controlled zone of abrasion, cutting, etc. The external tube 20 is maintained in a selected position relative to the burr 10 by stand-offs 21. These may be of any appropriate design, and are preferably constructed to tolerate at least intermittent contact with the rotating hollow shaft 40. In the design as illustrated, the stand-offs 21 are preferably configured to minimize fluid flow past the standoffs and into the volume 60 between the external tube 20 and the shaft 40. Removal of fluid and debris is accomplished through one or more openings 65 in the distal end of the hollow shaft 40, such that the fluid flows through lumen 67 in the support tube 30 to an exit at 66.

Because the debris is typically small in diameter, and the outlet space 67 is relatively large, suction is not typically required for debris removal with either of the above-described embodiments of this design. In particular, a vacuum source or a suction or aspiration source is not typically needed. A slight positive pressure, for example provided by elevation of a bag of saline solution used for irrigating the site, can be sufficient to provide flow through the outlet space 67 or 60. As previously mentioned, some debris-removal impulse can also be provided by the design of the burr 10 even in the absence of a hydrostatic head in the operation site.

An additional improvement provided by this design is the ability, in some embodiments, to vary the position of the external tube 20 with respect to the burr 10, by sliding or rotating the external tube 20 with respect to the shaft 40. A simple bellows or tight concentric shells (e.g., as in a radio antenna; not illustrated) at the proximal end of the external tube 20 would supply the needed range of motion. Movement of the external tube 20 can be by hand, as the proximal region of the external tube 20 near the handpiece body 90 is normally outside of the entry point into the patient; or mechanical or other controls can be provided.

An additional advantage of the design is that the external tube 20 can be made of plastic, allowing direct visual or fluoroscopic observation of the position of the abrader. The tip of the external tube 20 can be made to be radio-opaque or visible (e.g., by dye) if desired.

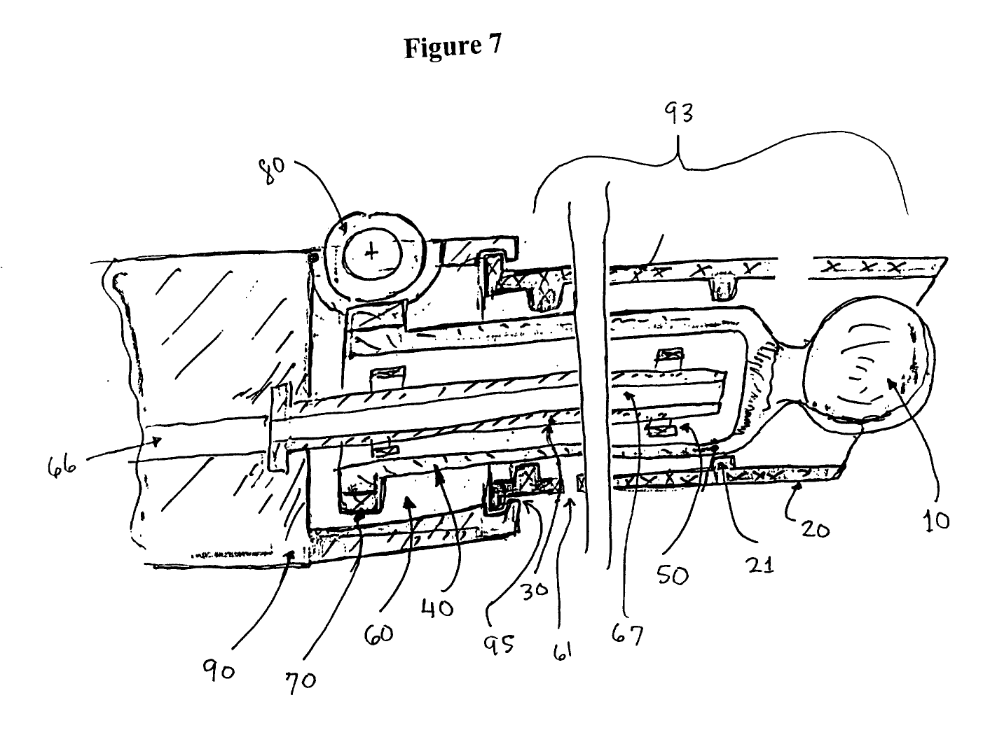

FIG. 7 shows a cross section of an embodiment similar to that of FIG. 6. In FIG. 7, the standoffs 21 can be longitudinal ribs or discrete “feet” as previously described, so that most of the annular area 60 bounded by the external tube 20 and the shaft 40 is open. This allows the space between the external tube 20 and the shaft 40 to be used either as an outlet for debris, or an inlet for lavage of the site of operation, or both. Removal of the fluid may be through an opening 61 in the side of the external tube 20. (And, in contrast to FIG. 6, there would not need to be an opening 65 in the shaft 40, unless two separate fluid passage routes were desired, for example one for influx and one for efflux.) The opening 61 may be near the proximal end of external tube 20, as illustrated, or elsewhere on the tube. Otherwise, the embodiment illustrated in FIG. 7 is substantially identical to the embodiment illustrated in FIG. 6. A similar arrangement for debris removal may also be provided in certain arrangements of other embodiments of the invention, for example in certain arrangements of the embodiments as illustrated in FIGS. 3, 4, and 5.

While several embodiments of the invention have been described and illustrated herein, those of ordinary skill in the art will readily envision a variety of other means and structures for performing the functions and/or obtaining the results or advantages described herein, and each of such variations or modifications is deemed to be within the scope of the present invention. More generally, those skilled in the art would readily appreciate that all parameters, dimensions, materials, and configurations described herein are meant to be exemplary and that actual parameters, dimensions, materials, and configurations will depend upon specific applications for which the teachings of the present invention are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described. The present invention is directed to each individual feature, system, material and/or method described herein. In addition, any combination of two or more such features, systems, materials and/or methods, provided that such features, systems, materials and/or methods are not mutually inconsistent, is included within the scope of the present invention. In the claims (as well as in the specification above), all transitional phrases or phrases of inclusion, such as “comprising,” “including,” “carrying,” “having,” “containing,” “composed of,” “made of,” “formed of” and the like shall be interpreted to be open-ended, i.e. to mean “including but not limited to.” Only the transitional phrases or phrases of inclusion “consisting of” and “consisting essentially of” are to be interpreted as closed or semi-closed phrases, respectively, as set forth in MPEP section 2111.03.

What is claimed is:

1. A surgical device comprising:
a body providing a handle;
a shaft carrying a burr;
a motor drivingly coupled to the shaft;
a support element configured to provide support for the shaft and positioned so that at least a portion of said support element is located distally of the handle;
a bearing located distally of the handle and between the support element and the shaft;
an external tube connecting proximally to the handle, said external tube being distinct from said support element; and
a sheath element for protecting tissue adjacent the burr from the action of the burr, wherein
the sheath element is a distal portion of at least one of the support element and the external tube, and wherein
the external tube provides substantially no lateral support for the shaft or the burr.

2. The device of claim 1 wherein a space between the external tube and the outermost of the shaft carrying the burr, and the support element for the shaft, forms a portion of a path for passage of fluid to or from an operating site.

3. The device of claim 1 wherein the external tube has standoffs, the standoffs providing a fixed amount of separation between the external tube, and the outermost of the shaft and the support element for the shaft.

4. The device of claim 1, wherein the external tube provides the sheath element.

5. The device of claim 1, wherein the support element provides the sheath element.

6. The device of claim 5 wherein the support element is perforated in a sheath region to provide passage for fluid from the burr to the vicinity of the external tube.

7. The device of claim 1 wherein the support element terminates proximal of the burr.

8. The device of claim 1 wherein the support element is interior of the shaft.

9. The device of claim 8 wherein a path for passage of fluid to or from an operating site is provided via an opening in the shaft.

10. The device of claim 1 wherein the device is rendered suitable for endoscopic use by having an outer diameter of the external tube of about one inch or less.

11. The device of claim 1 wherein the device is constructed and configured such that removal of debris from a site of operation with the device does not require the application of an external vacuum to the device.

12. The device of claim 1, wherein the motor comprises a liquid jet powered rotor.

13. The device of claim 1, wherein the motor comprises a turbine.

14. The device of claim 1, wherein the motor comprises an electric motor.

15. A method for performing endoscopic surgery on a patient, the method comprising:
inserting an abrading instrument into the interior of a patient via one of a natural and an artificial opening;
abrading selected tissues interior of the patient; and
removing debris generated during the abrading step from the interior of the patient with the instrument;
wherein the instrument comprises a burr joined to a rotatable shaft, the shaft being laterally supported by a support tube or support shaft and a bearing disposed between the support tube or shaft and the shaft,
and wherein the instrument further comprises an external tube, separate from and at least partially surrounding the support shaft or tube and the shaft, which external tube is constructed and arranged so that debris removal occurs through a generally annular space formed between the external tube and the outer surface of the shaft and/or between the external tube and the support element.

16. A shaft assembly configured for use in a surgical instrument providing a rotatable shaft, the shaft assembly comprising:
a rotatable shaft;
a burr drivable by the shaft;
a support element configured to provide lateral support for the shaft;
a distal bearing positioned between the support element and the shaft; and
an external tube positioned to at least partially surround at least a portion of the support element, wherein
the external tube is configured and positioned with respect to the support element and shaft so that it provides substantially no lateral support for the shaft or the burr when the assembly is assembled in an operable configuration with the surgical instrument.
